(19) United States
(12) Patent Application Publication
(10) Pub. No.: US 2007/0197643 A1
(43) Pub. Date: Aug. 23, 2007

(54) USE OF (Z)-2-CYANO-3-HYDROXY-BUT-2-ENOIC ACID-(4'-TRIFLUOROMETHYLPHENYL)-AMIDE FOR TREATING INFLAMMATORY BOWEL DISEASE

(75) Inventors: Richard D. SCHEYER, Branchburg, NJ (US); Scot D. STYREN, Niantic, CT (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/693,358

(22) Filed: Mar. 29, 2007

Related U.S. Application Data
(63) Continuation of application No. PCT/US05/37161, filed on Oct. 18, 2005.
(60) Provisional application No. 60/620,255, filed on Oct. 19, 2004.

(51) Int. Cl. A61K 31/277

(52) U.S. Cl. 514/521

(57) ABSTRACT

The invention relates to the use of compound of Formula I in treating patients for the symptoms of inflammatory bowel disease including Crohn's disease and ulcerative colitis.

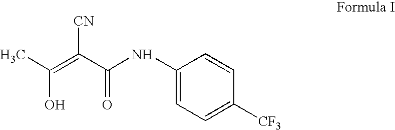

### CROSS-REFERENCE TO RELATED APPLICATIONS

This application is a continuation of International Patent Application No. PCT/US2005/037161 filed on Oct. 18, 2005 which is incorporated herein by reference in its entirety which also claims the benefit of priority of U.S. Provisional Appln. Ser. No. 60/620,255 filed on Oct. 19, 2004

### FIELD OF THE INVENTION

The present invention relates to methods of treating inflammatory bowel disease. In particular, the present invention relates to the treatment of inflammatory bowel disease with (Z)-2-cyano-3-hydroxy-but-2-enoic acid-(4′-trifluorom ethylphenyl)-amide, commonly known as teriflunomide.

### BACKGROUND OF THE INVENTION

(Z)-2-cyano-3-hydroxy-but-2-enoic acid-(4′-trifluoromethylphenyl)-amide (teriflunomide) has the structure illustrated in Formula I:

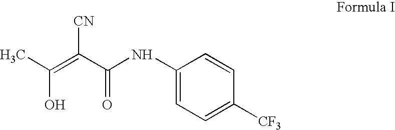

Generically known as teriflunomide the compound is an active metabolite of the disease-modifying, anti-rheumatic drug 5-methylisoxazole-4-carboxylic-(4-trifluoromethyl)-anilide commonly known as leflunomide, the structure of which is shown in Formula II. Leflunomide was first disclosed generically in U.S. Pat. No. 4,087,535 to Heubach, issued on May 2, 1978 and specifically in U.S. Pat. No. 4,284,786 to Kammerer et. al., issued on Aug. 18, 1981, wherein it was disclosed that the compound could be used for the treatment of multiple sclerosis. Also, the successful use of leflunomide for the treatment of Crohn's disease in a small number of patients, with azathioprine intolerance has been described (Prajapati, D. N., et al., 2001, American Journal of Gastroenterology, 96 (9): S305). Both of the aforementioned patents are incorporated herein by reference in their entirety.

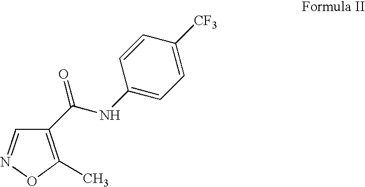

(Z)-2-cyano-3-hydroxy-but-2-enoic acid-(4′-trifluoromethylphenyl)-amide (teriflunomide, Formula I) and its' use in treating chronic graft-versus-host disease has been disclosed in U.S. Pat. No. 4,965,276 to Bartlett et. al. issued on Oct. 23, 1990. U.S. Pat. No. 5,459,163 also to Bartlett et. al. issued on Oct. 21, 1997 and U.S. Pat. No. 5,679,709 to Bartlett et. al. issued on Oct. 21, 1997 disclose compositions useful for treating autoimmune diseases in particular lupus erythematosus. Both of the aforementioned patents are incorporated herein by reference in their entirety. Teriflunomide has been shown to produce antiproliferative effects on a wide variety of immune cells and cell lines (Cherwinski H. M., et al., J. Pharmacol. Exp. Ther. 1995;272:460-8; Prakash A., et al., Drugs 1999;58(6):1137-66; Bartlett R. R. et al., Agent Action 1991;32(1-2):10-21) and anti-inflammatory activity in an animal models of inflammation (Huang, W-H. et al. Chem. Pharm. Bull., 2003, 51(3): 313-314 and U.S. Pat. No. 6,716,411 also to Bartlett et. al. issued on Apr. 6, 2004). Additionally, it inhibits the enzyme dihydrooate dehydrogenase, an enzyme essential for the synthesis of pyrimidines (Bruneau J-M, et al., Biochem. J. 1998; 36:299-303).

Inflammatory bowel disease (IBD) is a general term used to identify a series of related intestinal diseases of unknown etiology characterized by chronic inflammation at various portions of the gastrointestinal tract. Representative of IBD are Crohn's disease, ulcerative colitis, indeterminate colitis, and infectious colitis.

Crohn's disease is an idiopathic chronic enteritis of unknown etiology. This disease occurs most frequently in human beings of both sexes in their twenties and becomes chronic. It is a granulomatous lesion with fibrosis or ulceration and may be present within the whole alimentary tract from mouth to anus. The clinical symptoms of Crohn's disease are celialgia, general malaise, diarrhea, melena and occult bleeding positive, fervescence, loss of body weight, anemia, ileus, abdominal tumor and peritonitis.

Ulcerative colitis is an unaccountable disease of diffuse non-specific inflammation of the colon, which attacks the mucous membrane and often forms an erosion or ulcer. The lesion is chiefly sub-mucosal. The clinical symptoms of this disease are viscous-hemafecia, celialgia, hemafecia, watery stool, fervescence, loss of appetite, nausea and vomiting. Also, ulcerative colitis may be attended by such troubles as arthritis, stricture of the large intestine and copious bleeding, but their incidence is not high.

Current therapy for IBD includes anti-inflammatory drugs, immunosuppressive drugs and surgery. Sulfasalazine and related drugs having the bioactive 5-amino-salicylic acid (5-ASA) moiety are widely used to control moderate IBD symptoms and to maintain remission. Severe inflammation is often treated with corticosteroids and sometimes ACTH or with immunosuppressants such as 6-mercaptopurine, azathioprine, cyclosporine, and methotrexate. The most common surgical treatments for severe chronic IBD are intestinal resections and, ultimately, colostomy, which is a complete cure only for ulcerative colitis.

Severe side effects are associated with the drugs commonly prescribed for IBD, including nausea, dizziness, changes in blood chemistry (including anemia and leukopenia), skin rashes and drug dependence; and the surgical treatments are radical procedures that often profoundly alter the everyday life of the patient. Clearly there is a need for novel agents for the treatment of IBD without some of the side-effect liabilities associated with current therapy.

Teriflunomide itself has demonstrated efficacy in preclinical models of multiple sclerosis, and is in clinical trials for treatment of multiple sclerosis (MS). The pathophysiology of MS (R. A. Adams, M. V. Victor and A. H. Ropper eds., Principles of Neurology, McGraw-Hill, New York, 1997, pp. 903-921.) and IBD (D. K. Poldosky, N. Eng. J. Med., 347, 6: 417-429) disorders is mediated substantially via T-lymphocytes, the activation of which is modulated by teriflunomide. Also, there is some evidence suggesting partial overlap of MS with inflammatory bowel disease. (Purrman J et al. J. Clin. Gastroenterol. 1992, 14, 1: 43-46 and Kimura K. Mayo Clin. Proc. 2000, 75, 8: 802-806).

Following administration of teriflunomide, high amounts of teriflunomide enter the intestinal lumen. This may be due to either enterohepatic recirculation or via the intestinal mucosa. The intestinal mucosa is therefore exposed to relatively high amounts of teriflunomide, and therapeutic effects may be achieved at lower doses than those required to treat systemic autoimmune disease. Alterations in the integrity of the GI mucosa may provide further enhancement of local exposure. Thus, based upon the above discussions along with the good safety profile of teriflunomide, even at doses for treating systemic diseases, and coupled with the low likelihood of gastrointestinal side-effects, teriflunomide is clearly a potentially useful drug for the treatment of IBD, which includes Crohn's disease, ulcerative colitis, indeterminate colitis and infectious colitis.

### SUMMARY OF THE INVENTION

The present invention is a method of treating inflammatory bowel disease in patients which includes Crohn's disease, ulcerative colitis, indeterminate colitis and infectious colitis and the systemic conditions arising therefrom. The method comprises the administration of a compound of Formula I or a pharmaceutically acceptable salt thereof, formulated with other pharmaceutically acceptable excipients and carriers in a therapeutically effective amount to treat the disease.

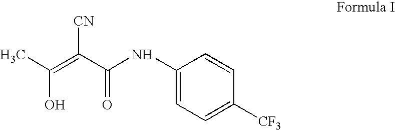

### DETAILED DESCRIPTION OF THE DRAWING

FIG. 1 shows the effect of teriflunomide on symptoms in the Rat Experimental Allergic Encephalomyelitis (EAE) at 3 different doses when administered orally (p.o.) as compared to vehicle and dexamethasone.

### DETAILED DESCRIPTION OF THE INVENTION

Teriflunomide and formulations comprising teriflunomide for the treatment of inflammatory bowel disease and its' symptomatic ramifications that are the basis for the present invention are herein described according to standard pharmacological terminology. Terms used herein have the meanings defined in this specification as follows.
a) “Pharmaceutically acceptable salts” means either an acid addition salt or a basic addition salt, whichever is possible to make with the compounds of the present invention.

“Pharmaceutically acceptable acid addition salt” is any non-toxic organic or inorganic acid addition salt of the base compounds represented by Formula I. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points.

“Pharmaceutically acceptable basic addition salts” means non-toxic organic or inorganic basic addition salts of the compounds of Formula I. Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.
b) “Patient” means a warm blooded animal, such as for example rat, mice, dogs, cats, guinea pigs, and primates such as humans.
c) “Treat” or “treating” means any treatment, including, but not limited to, alleviating symptoms, eliminating the causation of the symptoms either on a temporary or permanent basis, or preventing or slowing the appearance of symptoms and progression of the named disorder or condition.
d) “Therapeutically effective amount” means an amount of the compound, which is effective in treating the named disorder or condition.
e) “Pharmaceutically acceptable carrier” is a non-toxic solvent, dispersant, excipient, adjuvant or other material which is mixed with the compound of the present invention in order to permit the formation of a pharmaceutical composition, i.e., a dosage form capable of administration to the patient. One example of such a carrier is pharmaceutically acceptable oil typically used for parenteral administration.
f) “Stereoisomers” is a general term for all isomers of the individual molecules that differ only in the orientation of their atoms in space. It includes mirror image isomers (enantiomers), geometric (cis/trans) isomers, and isomers of compounds with more than one chiral center that are not mirror images of one another (diastereoisomers).
g) “Leflunomide” is the generic name for 5-methylisoxazole-4-carboxylic-(4-trifluoromethyl)-anilide.
h) “Teriflunomide ” is the generic name for (Z)-2-cyano-3-hydroxy-but-2-enoic acid-(4′-trifluoromethylphenyl)-amide.

The synthesis of the compound of Formula 1 has been disclosed, and is accomplished by methods that are well known to those skilled in the art. For example, U.S. Pat. No. 5,504,084 to Bartlett et. al. issued on Apr. 2, 1996, and U.S. Pat. No. 5,990,141 to Hirth et. al., issued on Nov. 23, 1999 disclose methods for the synthesis of teriflunomide. The aforementioned patents are incorporated herein by reference. Hirth et. al. '141 discloses the synthetic reaction scheme as illustrated in Scheme 1.

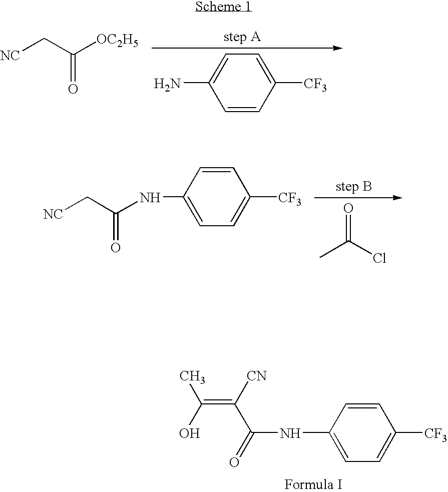

In Scheme 1, step A, commercially available cyanoacetic acid ethyl ester is reacted with commercially available 4-trifluoromethylaniline neat at elevated temperature to give cyanoacetic-(4-trifluoromethyl)anilide. In step B, the product from step A is dissolved in tetrahydrofuran and reacted with NaH in acetonitrile followed by reaction with acetyl chloride to produce the compound of Formula I.

Thus in accordance with the practice of the present invention, a method of treating inflammatory bowel disease is disclosed which comprises administering to a patient having said disease a therapeutically effective amount of a compound of Formula I, its stereoisomer, or a pharmaceutically acceptable salt thereof, optionally in combination with a pharmaceutically acceptable carrier

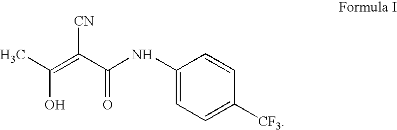

In another embodiment of the method of this invention said inflammatory bowel disease is Crohn's disease.

In a further embodiment of the method of this invention, said inflammatory bowel disease is ulcerative colitis.

In yet another embodiment of the method of this invention, said inflammatory bowel disease is indeterminate colitis.

In still another embodiment of the method of this invention, said inflammatory bowel disease is infectious colitis.

In a further embodiment of the method of this invention, said therapeutically effective amount of compound is an amount less than that required to treat systemic autoimmune diseases.

In another embodiment of the method of this invention, said therapeutically effective amount of compound is an amount less than about 10 mg/kg/day.

In yet another embodiment of the method of this invention, said therapeutically effective amount of compound is an amount from less than 1.0 mg/kg/day to about 10 mg/kg/day.

In treating a patient afflicted with a condition described above, a compound of Formula I can be administered in any form or mode which makes the compound bioavailable in therapeutically effective amounts, including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18th Edition, Mack Publishing Co. (1990), incorporated herein by reference.

The compound of the present invention may be administered orally, for example, in the form of tablets, troches, capsules, elixirs, suspensions, solutions, syrups, wafers, chewing gums and the like and may contain one or more of the following adjuvants: binders such as microcrystalline cellulose, gum tragacanth or gelatin; excipients such as starch or lactose, disintegrating agents such as alginic acid, Primogel®, corn starch and the like; lubricants such as magnesium stearate or Sterotex®; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials, which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors.

The compound of Formula I of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The dosage range at which the compound of Formula I exhibits its ability to act therapeutically can vary depending upon its severity, the patient, the formulation, other underlying disease states that the patient is suffering from, and other medications that may be concurrently administered to the patient. Generally, the compound of Formula I will exhibit their therapeutic activities at dosages of between about 0.001 mg/kg of patient body weight/day to about 100 mg/kg of patient body weight/day.

All of the references described herein are incorporated herein by reference in their entirety. The examples detailed below are provided to better describe and more specifically set forth the compounds, processes and methods of the present invention. It is to be recognized that they are for illustrative purposes only however, and should not be interpreted as limiting the spirit and scope of the invention as later recited by the claims that follow.

### EXAMPLE 1

### Rat Experimental Allergic Encephalomyelitis (Rat EAE)

This example illustrates a method for studying the inflammation of the brain and spinal cord associated with multiple sclerosis (MS), a T-cell related autoimmune disease. See Bolton, C. Multr. Scler. 1995; 1(3); 143-9.

Experimental allergic encephalomyelitis (EAE) is a T-cell-mediated autoimmune disease of the nervous system that develops in susceptible animals following sensitization with either whole spinal cord homogenate or a component (myelin basic protein). The EAE rodent model is an appropriate tool for studying the inflammation of the brain and spinal cord observed in MS patients. In rodents, injection of whole spinal cord or spinal cord components such as myelin basic protein induces an autoimmune response based on the activation of T-lymphocytes. Clinical disease typically becomes manifest around day 8-10 after inoculation, observed as a broad spectrum of behavioral anomalies ranging from mild gait disturbances and tail atony to complete paralysis and death. Weight loss typically occurs. In animals that survive, spontaneous recovery occurs, accompanied by variable recovery of most motor function. Depending on the species, allergen, and methodology used, animals tested by the EAE model may experience a single (acute EAE) or several (chronic relapsing EAE) attacks. Several treatment paradigms may be used: the drug or treatment of choice may be administered before immunization, during the non-symptomatic period or during the clinical disease.

Animals:

Female Lewis rats, 160-220 g (Charles River)

Antigen:

Whole Guinea Pig spinal cord (Harlan Biosciences).

Complete Freund's adiuvant H37 Ra [1 mg/ml Mycobacterium Tuberculosis H37 Ra] (Difco).

Additional Antigen:

Mycobacterium Tuberculosis (Difco).

Bordetella Pertussis [Heat Killed] (Difco).
Antigen preparation: (for approximately 720 animals)
   1. Weigh 5 g of frozen guinea pig spinal cord.
   2. Add 5 g spinal cord to 5 ml 0.9% saline (1 g/ml) in a round bottom centrifuge tube
   3. Homogenize on ice with the Tissue-tech until the tissue is completely disrupted (approximately 5 minutes).
   4. Add 10 ml Complete Freund's adjuvant H37 Ra supplemented with 200 mg Mycobacterium Tuberculosis (20 mg/ml Complete Freund's adjuvant H37 Ra).
   5. Extract homogenate/adjuvant from tube by sucking it into a 10 ml syringe fitted with an 18 gauge emulsifying needle.
   6. Emulsify between two 30 ml glass syringes until it becomes difficult to continue passing the material through the needle. (Approximately 5 min {there must be no separation between the oil phase and the aqueous phase}).
   7. Use immediately or keep on ice until needed (not more than 30 min) (do not freeze).

### Protocol

1. Female Lewis rats (Charles River) are given free access to food and water and should be acclimated a minimum of 3 days before use in experiments.
2. Rats weighing 160 and 220 g are initially induced with 5% isoflurane (Aerrane, Fort Dodge), 30% O₂, 70% N₂O for 2-5 minutes.
3. The rat is then placed onto a circulating water heating blanket (Gaymar) (dorsal surface up) and into the nose cone for spontaneous respiration of anesthetic gases. The isoflurane is reduced to 2%.
4. Two subcutaneous injections (0.1 ml each) of either antigen or normal saline are made into ventral surface of the hind paws.
5. The animals are removed from the nose cone, weighed and numbered.
6. The rats are allowed to awake from anesthesia and are placed into individual cages.
7. The animals are observed daily for signs of EAE induction (see criteria below)
STAGE:0 NORMAL
STAGE 1 Abnormal gate and tail atony
STAGE 2 Mild but definite weakness of one or both hind legs
STAGE: 3 Severe weakness of one or both hind legs or mild ataxia
STAGE: 4 Severe paraparesis and minimal hind leg movement
STAGE: 5 No hind leg movement and paraplegia
STAGE: 6 Moribund state with no spontaneous movement and impaired respiration.
   Increasing degree of front leg involvement and urinary and fecal incontinence may also occur
STAGE:7 DEATH

Treatment was begun on day 10 after immunization. Since the disease symptoms in this model typically appear 10-11 days after inoculation, this time point may be considered to represent the initial phase of an acute episode of MS. It is judged that this delay of the start of treatment mimics the clinical situation more closely than the traditionally used protocols where drugs are administered at the time of, or even before, inoculation (Teitelbaum D. et al., Proc Natl Acad Sci USA 1999; 96: 3842-3847 and Brod S. A., et al., Ann Neurol 2000; 47: 127-131).

The effect of teriflunomide on symptoms of EAE in rat at various doses is illustrated in FIG. 1. Dexamethasone is included in the figure for comparison.

### EXAMPLE 2

### 2,4-Dinitrobenzenesulfonic Acid (DNBS) Induced Distal Colitis

This Example illustrates the anti-inflammatory activity of one compound of this invention using a model of 2,4-dinitrobenzenesulfonic acid (DNBS) induced distal colitis (a model of inflammatory bowel disease).

Use groups of 3 male or female rats fasted for 24 hours. Induce distal colitis by intra-colonic instillation of DNBS (2,4-dinitrobenzene sulfonic acid, 30 mg in 0.5 ml ethanol 30%) after which gently inject air (2 ml) through the cannula to ensure that the solution remains in the colon. Administer Test substance PO (30 mg/kg) at 24 and 2 hours before DNBS instillation. Then, the animals receive test compound every 24 hours for 5 consecutive days. Give the control group vehicle alone as compound dosing pattern. Sacrifice the animals 24 hours after the final dose of test compound administration and remove each colon and weigh. Obtain colon-to-body weight ratio from the percentage of the comparison between the animal colon weight and body weight. A 30 percent or more (.+−.30%) reduction in colon-to-body weight ratio relative to the vehicle treated control group was considered significant. See C. M. Hogaboam et al. Eur. J. Pharmacol. 309:261 (1996).

We claim:

1. A method of treating inflammatory bowel disease which comprises administering to a patient having said disease a therapeutically effective amount of a compound of Formula I, its stereoisomer, or a pharmaceutically acceptable salt thereof, optionally in combination with a pharmaceutically acceptable carrier,

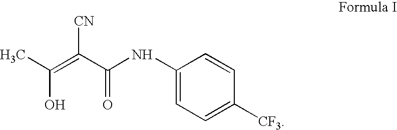

2. The method of claim 1 wherein said inflammatory bowel disease is Crohn's disease.

3. The method of claim 1 wherein said inflammatory bowel disease is ulcerative colitis.

4. The method of claim 1 wherein said inflammatory bowel disease is indeterminate colitis.

5. The method of claim 1 wherein said inflammatory bowel disease is infectious colitis.

6. The method of claim 1 wherein said administration to the patient is delivered by a means selected from the group consisting of orally, sublingually, buccally, subcutaneously, intramuscularly, intravenously, transdermally, intranasally, rectally, and topically.

7. The method of claim 6 wherein said pharmaceutically acceptable carrier is selected from the group consisting of binders, dissolution agents, bulking agents, lubricants, solvents and solutions, tablefting agents, disintegrating agents, glidants flavor agents, sweeteners, coloring agents, stabilizers, pH-adjusting agents and mixtures thereof.

8. The method of claim 7 wherein said pharmaceutically acceptable carrier is selected from the group consisting of microcrystalline cellulose and other cellulose derivatives, natural and synthetic gums and gum derivatives, sugar alcohols, polyethylene glycol, oils, polyvinylpyrrolidone (PVP), gelatin, wax and wax derivatives, starch, lactose, alginic acid, corn starch and starch derivatives, magnesium stearate, colloidal silicon dioxide; sucrose, aspartame, sucralose, saccharin, peppermint, methyl salicylate or orange flavoring.
